(19) United States
(12) Patent Application Publication
(10) Pub. No.: US 2004/0097587 A1
(43) Pub. Date: May 20, 2004

(54) Compositions and methods of treatment of ulcerating diseases, burns, and related conditions

(76) Inventor: Jack L Arbiser, Atlanta, GA (US)

Correspondence Address: Christopher B Linder, Thomas Kayden Horstemeyer & Risley, Suite 1750, 100 Galleria Parkway NW, Atlanta, GA, 30339-5948, US

(21) Appl. No.: 10/473,171

(22) PCT Filed: Mar. 27, 2002
(86) PCT No.: PCT/US02/09527

(51) Int. Cl. A61K 31/203

(52) U.S. Cl. 514/559

(57) ABSTRACT

Pharmaceutical compositions and methods of treating ulcerating diseases, burns, and related conditions are disclosed. The methods include administering to a host in need of treatment an effective amount of at least one retinoid receptor compound. In addition, the pharmaceutical compositions include at least one retinoid receptor compound in combination with a pharmaceutically acceptable carrier. The retinoid receptor compound can be present in a dosage level effective to treat conditions listed above.

### CROSS-REFERENCE TO RELATED APPLICATION

This application claims priority to copending U.S. provisional application entitled, “Simulation Of Wound And Ulcer Healing By Retinoids,” having ser. No. 60/279093, filed Mar. 27, 2001, which is entirely incorporated herein by reference.

### TECHNICAL FIELD

The present invention is generally related to compositions and methods for administration to hosts and, more particularly, is related to compositions designed for treatment of ulcerating diseases, burns, and related conditions and methods of administration thereof.

### BACKGROUND

Venous ulcers are a common and debilitating disease that costs approximately three to four billion dollars a year to treat. Typically, ulcerating diseases, such as venous ulcers, are exhibited by lesions on the skin or mucous membranes marked by inflammation, necrosis, and/or sloughing of damaged tissues. In particular, venous ulcers are characterized by brown discoloration of the skin as well as swelling, pain, redness, and dry, itchy skin. Patients typically have venous ulcers on the medial (inner aspect) of the distal leg or ankle area, but venous ulcers may occur in other areas of the body as well.

All veins have one-way valves to assist blood flow toward the heart. Venous dysfunction occurs when the forward flow of venous blood is significantly impaired. Therefore, damage to valves or loss of valvular function results in venous hypertension, venous stasis, perfusion of tissues with de-oxygenated blood, and finally venous ulcers. Risk factors for developing venous insufficiency (inadequate venous function), and ultimately a venous ulcer, include varicose veins, deep venous disease, incompetent perforating veins, and post-thrombotic syndrome. However, there are no universally accepted treatments for venous ulcers.

Thus, a heretofore unaddressed need exists in the industry to address at least the aforementioned deficiencies and/or inadequacies in regard to treating ulcerating diseases, burns, and related conditions.

### SUMMARY OF THE INVENTION

Briefly described, embodiments of the present invention include representative methods to treat ulcerating diseases, burns, and related conditions. A representative method includes administering to a host in need of treatment an effective amount of at least one retinoid receptor compound.

Alternate embodiments of the present invention also include pharmaceutical compositions having at least one retinoid receptor compound in combination with a pharmaceutically acceptable carrier. The retinoid receptor compound is present in a dosage level effective to treat ulcerating diseases, burns, and related conditions.

Other systems, methods, features, and advantages of the present invention will be or will become apparent to one with skill in the art upon examination of the following detailed description. It is intended that all such additional systems, methods, features, and advantages be included within this description, be within the scope of the present invention, and be protected by the accompanying claims.

### DETAILED DESCRIPTION

The present invention provides for compositions and methods of treating hosts having ulcerating diseases, burns, and related conditions. More particularly, embodiments of the present invention include compositions having at least one retinoid receptor compound that can be used to treat one or more of the conditions discussed herein.

In general, retinoids play important roles in a variety of biological phenomena. Retinoids are important because they interact with two families of nuclear receptors, called retinoic acid receptors (RARs) and retinoid X (rexinoid) receptors (RXRs). RARs and RXRs mediate pharmacological and physiological retinoid signaling. In particular, RARs and RXRs are ligand-dependent transcription factors, which regulate gene expression in at least two different ways. First, RARs and RXRs can up-regulate the expression of genes by binding to the RA-responsive elements (RAREs) present in their promoters. Second, RARs and RXRs can down-regulate the expression of genes by antagonizing the enhancer action of certain other transcription factors.

The distinct isotypes of RARs (alpha, beta, and gamma) and RXRs (alpha, beta, and gamma) are encoded by six separate genes. Each RAR isotype is further expressed as several isoforms differing in their N-terminal A region, which are generated by alternative splicing and/or by differential usage of more than one promotor. RAR(alpha) is expressed as two main isoforms (alpha₁ and alpha₂), RAR(beta) as four isoforms (beta₁, beta₂, beta₃, and beta₄), and RAR(gamma) as two main isoforms (gamma₁ and gamma₂).

The two families of retinoid receptors differ from each other with respect to the ligands that bind and activate the receptors. For example, all-trans-RA (RA) binds and activates the RAR family of receptors, while 9-cis-RA (9C-RA) binds and activates both the RARs and members of the RXR family.

Compounds having retinoid-like activity can be useful for treating animals of the mammalian species, including humans, and for curing and/or alleviating the symptoms and conditions of numerous diseases and conditions. In other words, compositions having one or more retinoid-like compounds as the active ingredient may be useful as regulators of cell proliferation and differentiation.

In general, retinoid receptor compounds can be used to treat a condition because they can activate transcription via the retinoid receptors (e.g., RAR and RXR, as well as their homodimers and heterodimers). Retinoid receptor compounds can include all natural and/or synthetic analogs of Vitamin A, or retinol-like compounds which possess the biological activity of Vitamin A, in the skin as well as the geometric isomers and stereoisomers of these compounds.

In particular, retinoid receptor compounds can include natural retinoid compounds, synthetic retinoid compounds, natural rexinoid compounds, and synthetic rexinoid compounds that can activate transcription via retinoid receptors. Natural retenoids include isoprenoids that contain or are derived from four prenyl groups (H—[CH₂C(CH₃)CHCH₂]₄—) linked head-to-tail. Synthetic retinoids (e.g., arotinoids and heteroarotinoids) may not have a retinoid chemical structure, but nevertheless act in a similar biological manner as natural retinoids.

In particular, retinoid receptor compounds can include, but are not limited to, retinoic acid, tretinoin (all-trans-retinoic acid) and isotretinoin (13-cis-retinoic acid), 9-cis retinoic acid, adapalene, retinol, retinol esters (e.g., retinyl palmitate, retinyl acetate, retinyl propionate), tocopheryl-retinoate, adapalene, azelaic acid, motretinide, bexarotene, and tazarotene. In addition, retinoid receptor compounds can include peroxisome proliferator-activated receptor (PPAR) ligands such as, but not limited to, PPAR(alpha), PPAR(gamma), PPAR(delta), fibrates, and thiazolidinediones.

Where such forms exist, retinoid receptor compounds can include retinoid receptor compound analogues, retinoid receptor compound homologues, retinoid receptor compound isomers, or retinoid receptor compound derivatives thereof, that can function in a similar biological manner as retinoid receptor compounds to treat ulcerating diseases, burns, and related conditions in a host. In addition, retinoid receptor compounds can include pharmaceutically acceptable salts, esters, and prodrugs of the retinoid receptor compounds described or referred to above.

As indicated above, compositions of this invention can be used to treat ulcerating diseases. In general, ulcerating diseases can include lesions on the skin or mucous membranes marked by inflammation, necrosis, and/or sloughing of damaged tissues. A variety of conditions can cause ulcers, such as trauma, caustic chemicals, intense heat or cold, arterial or venous stasis, cancers, drugs, infectious agents, etc. In particular, ulcerating diseases include, but are not limited to, aphthous ulcers, venous ulcers, diabetic ulcers, arterial ulcers, decubitus ulcers, etc.

In addition, compositions of this invention can be used to treat burns. Burns can include thermal burns, chemical burns, electrical burns, and radioactive burns, for example. Compositions of this invention can be used to treat ulcerating diseases, burns, and related conditions at any stage from the discovery of the condition to advanced stages of the condition.

By “pharmaceutically acceptable salt” it is meant those salts which are, within the scope of sound medical judgement, suitable for use in contact with the tissues of hosts without undue toxicity, irritation, allergic response and the like, are commensurate with a reasonable benefit/risk ratio, and are effective for their intended use. The salts can be prepared in situ during the final isolation and purification of one or more compounds of the composition, or separately by reacting the free base function with a suitable organic acid.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

Representative alkali or alkaline earth metal salts that may be used as the pharmaceutically acceptable salts include, but are not limited to, sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pharmaceutically acceptable esters” as used herein refers to those esters of one or more compounds of the composition which are, within the scope of sound medical judgement, suitable for use in contact with the tissues of hosts without undue toxicity, irritation, allergic response, and the like, are commensurate with a reasonable benefit/risk ratio, and are effective for their intended use.

The term “pharmaceutically acceptable prodrugs” as used herein refers to those prodrugs of one or more compounds of the composition which are, within the scope of sound medical judgement, suitable for use in contact with the tissues of hosts without undue toxicity, irritation, allergic response, and the like, are commensurate with a reasonable benefit/risk ratio, and are effective for their intended use. Pharmaceutically acceptable prodrugs also include zwitterionic forms, where possible, of one or more compounds of the composition. The term “prodrug” refers to compounds that are rapidly transformed in vivo to yield the parent compound, for example by hydrolysis in blood.

Compositions of this invention may be suitable for oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, intravesical, and epidural) administration.

The compositions may conveniently be presented in unit dosage form and may be prepared by conventional pharmaceutical techniques. Such techniques include the step of bringing into association one or more compositions of the present invention and one or more pharmaceutical carriers or excipients.

Compositions of the present invention suitable for oral administration may be presented as discrete units such as, but not limited to, tablets, caplets, pills or dragees, capsules, or cachets, each containing a predetermined amount of one or more of the compositions; as a powder or granules; as a solution or a suspension in an aqueous liquid or a non-aqueous liquid; or as an oil-in-water liquid emulsion or a water-in-oil emulsion, or as a bolus, etc.

Compositions of the present invention suitable for topical administration in the mouth include for example, lozenges, having the ingredients in a flavored basis, usually sucrose and acacia or tragacanth; pastilles, having one or more of the compositions of the present invention in an inert basis such as gelatin and glycerin, or sucrose and acacia; and mouthwashes, having one or more of the compositions of the present invention administered in a suitable liquid carrier.

Compositions of the present invention suitable for topical administration to the skin may be presented as ointments, creams, gels, and pastes, having one or more of the compositions administered in a pharmaceutical acceptable carrier.

Compositions of the present invention for rectal administration may be presented as a suppository with a suitable base comprising, for example, cocoa butter or a salicylate.

Compositions of the present invention suitable for nasal administration, when the carrier is a solid, include a coarse powder having a particle size, for example, in the range of 20 to 500 microns which is administered in the manner in which snuff is taken, (i~e., by rapid inhalation through the nasal passage from a container of the powder held close up to the nose). When the carrier is a liquid (for example, a nasal spray or as nasal drops), one or more of the compositions can be admixed in an aqueous or oily solution, and inhaled or sprayed into the nasal passage.

Compositions of the present invention suitable for vaginal administration may be presented as pessaries, tampons, creams, gels, pastes, foams or spray formulations containing one or more of the compositions and appropriate carriers.

Compositions of the present invention suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The compositions may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

Compositions of the present invention may be used as the active ingredient in combination with one or more pharmaceutically acceptable carrier mediums and/or excipients. As used herein, “pharmaceutically acceptable carrier medium” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, the compositions of the invention may be combined with pharmaceutically acceptable excipients, and, optionally, sustained-release matrices, such as biodegradable polymers, to form therapeutic compositions. A “pharmaceutically acceptable excipient” refers to a non-toxic solid, semi-solid or liquid filler, diluent, encapsulating material or formulation auxiliary of any type.

Except insofar as any conventional carrier medium is incompatible with the compounds used in practicing embodiments of the invention, such as by producing any undesirable biological effect or otherwise interacting in a deleterious manner with one or more of the compounds of the pharmaceutical composition, its use is contemplated to be within the scope of the embodiments of this invention.

When used in the above or other treatments, a therapeutically effective amount of one or more of the components of the compositions may be employed in pure form or, where such forms exist, in pharmaceutically acceptable salt, ester, and prodrug form. By a “therapeutically effective amount” of one or more of the components of the composition it is meant a sufficient amount of one or more of the components to treat ulcerating diseases, burns, and related conditions at a reasonable benefit/risk ratio applicable to any medical treatment.

It will be understood, however, that the total daily usage of the compositions of the present invention will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed; the age, body weight, general health, sex and diet of the patient; the time of administration; route of administration; rate of excretion of the specific compound employed; the duration of the treatment; drugs used in combination or coincidential with the specific composition employed; and like factors well known in the medical arts. For example, it is well within the skill of the art to start doses of the composition at levels lower than those required to achieve the desired therapeutic effect, and to gradually increase the dosage until the desired effect is achieved.

Compositions of the present inventions are preferably formulated in dosage unit form for ease of administration and uniformity of dosage. “Dosage unit form” as used herein refers to a physically discrete unit of the composition appropriate for the host to be treated. Each dosage should contain the quantity of composition calculated to produce the desired therapeutic affect either as such, or in association with, the selected pharmaceutical carrier medium.

Preferred unit dosage formulations are those containing a daily dose or unit, daily sub-dose, or an appropriate fraction thereof, of the administered ingredient. For example, significant improvement was observed in a female having venous ulcer and related conditions for five months after orally taking approximately 80 milligrams per day of isotretinoin for two months. In particular, the venous ulcer had healed, the lesions had improved, and the vessel prominence was significantly decreased, all of which are discussed in more detail in McLaughlin et al., J. Am. Acad. of Dermatol, 45, 462-465, which is herein incorporated by reference.

It should be noted that approximately 5 to 160 milligrams per day of one or more retinoid receptor compounds could be administered to a host to produce similar results. Similarly, it should be noted that approximately 0.1 to 2 milligrams of one or more retinoid receptor compounds per kilogram of the host per day could be administered to produce similar results. A therapeutically effective dose level will depend on many factors, as described above. In addition, it is well within the skill of the art to start doses of the composition at levels and increase the dosage until the desired effect is achieved.

Compositions of the present invention may be used in combination with other compositions and/or procedures (e.g., compression stockings, dressings, grafting silvadene, and unna boots) for the treatment of the conditions described above.

Compositions of the present invention may be used with a sustained-release matrix. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

As indicated above, compositions of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to one or more compositions of the present invention, stabilizers, preservatives, excipients, and the like. The preferred lipids are the phospholipids and the phosphatidyl cholines (lecithins), both natural and synthetic. Methods to form liposomes are known in the art.

As used herein, the term “host” includes both humans, mammals (e.g., cats, dogs, horses, etc.), and other living species that are in need of treatment. In addition, a “composition” can include one or more retinoid receptor compounds.

It should be emphasized that the above-described embodiments of the present invention are merely possible examples of implementations, and are set forth only for a clear understanding of the principles of the invention. Many variations and modifications may be made to the above-described embodiments of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A method to treat a condition chosen from an ulcerating disease and a burn comprising: administering to a host in need of treatment an effective amount of at least one retinoid receptor compound chosen from tretinoin and isotretinoin.

2. The method of claim 1, wherein the ulcerating disease is a venous ulcer.

3. The method of claim 1, wherein the ulcerating disease is an aphthous ulcer.

4. The method of claim 1, wherein the ulcerating disease is a diabetic ulcer.

5. The method of claim 1, wherein the ulcerating disease is a decubitus ulcer.

6. A method to treat an ulcerating disease comprising: administering to a host in need of treatment an effective amount of at least one retinoid receptor compound.

7. The method of claim 6, wherein the ulcerating disease is chosen from a venous ulcer, an aphthous ulcer, a diabetic ulcer, and a decubitus ulcer.

8. The method of claim 6, wherein the at least one retinoid receptor compound includes a retinoid compound.

9. The method of claim 6, wherein the at least one retinoid receptor compound includes a rexinoid compound.

10. The method of claim 6, wherein the at least one retinoid receptor compound includes peroxisome proliferator-activated receptor ligands.

11. The method of claim 6, wherein the at least one retinoid receptor compound includes pharmaceutically acceptable salts of the retinoid receptor compound.

12. The method of claim 6, wherein the at least one retinoid receptor compound includes pharmaceutically acceptable prodrugs of the retinoid receptor compound.

13. A method to treat a burn comprising: administering to a host in need of treatment an effective amount of at least one retinoid receptor compound.

14. The method of claim 13, wherein the at least one retinoid receptor compound includes a retinoid compound.

15. The method of claim 13, wherein the at least one retinoid receptor compound includes a rexinoid compound.

16. A pharmaceutical composition comprising: at least one retinoid receptor compound in combination with a pharmaceutically acceptable carrier, wherein the at least one retinoid receptor compound is present in a dosage level effective to treat a condition chosen from an ulcerating disease and a burn.

17. The pharmaceutical composition of claim 16, wherein the at least one retinoid receptor compound includes a retinoid compound.

18. The pharmaceutical composition of claim 16, wherein the at least one retinoid receptor compound includes a rexinoid compound.

19. The pharmaceutical composition of claim 16, wherein the at least one retinoid receptor compound includes peroxisome proliferator-activated receptor ligands.

20. The pharmaceutical composition of claim 16, wherein the at least one retinoid receptor compound includes isotretinoin and is administered orally.

21. The pharmaceutical composition of claim 16, wherein the at least one retinoid receptor compound includes tretinoin and is administered topically.

22. The pharmaceutical composition of claim 16, wherein the at least one retinoid compound includes pharmaceutically acceptable salts of the retinoid receptor compound.

23. The pharmaceutical composition of claim 16, wherein the at least one retinoid compound includes pharmaceutically acceptable prodrugs of the retinoid receptor compound.

24. The pharmaceutical composition of claim 16, wherein pharmaceutical composition can be administered orally, rectally, parenterally, intrasystemically, intravaginally, intraperitoneally, topically, and bucally.
